(19) United States
(12) Patent Application Publication
(10) Pub. No.: US 2010/0028912 A1
(43) Pub. Date: Feb. 4, 2010

(54) IMAGING USING RADIOACTIVE MONOCATIONS IN COMBINATION WITH A RECEPTOR BINDING LIGAND THAT STIMULATES THE INFLUX OF CATIONS

(75) Inventor: Karen E. Linder, Kingston, NJ (US)

Correspondence Address: BRACCO RESEARCH USA INC., 305- COLLEGE ROAD EAST, PRINCETON, NJ, 08540, US

(73) Assignee: BRACCO IMAGING S.P.A., Milan (IT)

(21) Appl. No.: 12/519,655

(22) PCT Filed: Dec. 18, 2007
(86) PCT No.: PCT/US07/25898

§ 371 Date: Jun. 17, 2009

Related U.S. Application Data
(60) Provisional application No. 60/875,884, filed on Dec. 20, 2006.

(51) Int. Cl. G01N 33/567

(52) U.S. Cl. 435/7.21

(57) ABSTRACT

A novel method of imaging, comprising administration of a radioactive mono-cation, such as, for example, ⁸²Rubidium (positron imaging) or Thallium (SPECT or planar imaging), in combination with a receptor binding ligand that stimulates the influx of cations. The method permits imaging of receptor-expressing tissue. However, unlike imaging methods which rely on a radiolabeled binding ligand, in the instant method the influx of the radioactive mono-cation is measured, not the binding of a radiolabeled ligand. Thus, there is a decreased likelihood of receptor saturation and potentially less radiation exposure for the patient.

### RELATED APPLICATIONS

This application claims priority to and benefit of U.S. Provisional application No. 60/875,884, filed Dec. 20, 2006, all of which are hereby incorporated by reference in its entirety.

### FIELD OF THE INVENTION

A novel method of imaging, using a radioactive mono-cation, such as, for example, ⁸²Rubidium (positron imaging) or ²⁰¹Thallium (SPECT or planar imaging), in combination with a receptor binding ligand that stimulates the influx of cations.

### BACKGROUND OF THE INVENTION

Both ⁸²Rubidium (⁸²Rb⁺) and ²⁰¹Thallium (²⁰¹Tl⁺) are currently used for nuclear imaging of the myocardium, and the resulting images provide valuable information with respect to both blood flow and tissue viability in clinical practice. Both ²⁰¹Tl and ⁸²Rb (and indeed all isotopes of Rb) are recognized as analogs for the potassium ion (K⁺) in vivo. When injected into a subject, these mono-cationic isotopes are taken up by myocardial tissue in proportion to blood flow. In areas with little or no blood flow, very little isotope is deposited in the myocardial tissue; in regions of high blood flow, higher amounts of the radioisotope localize in the tissue.

The distribution of the agents can be determined using nuclear imaging procedures that are well known in the art. For ²⁰¹Tl⁺, images are obtained with a gamma camera that is sensitive to the gamma photons that are emitted. For ⁸²Rb⁺, images are obtained using Positron Emission Tomography, or PET, as ⁸²Rb⁺ emits positrons. Since ⁸²Rb⁺ has a very short half-life (75 seconds), the ⁸²Rb⁺ used in imaging studies is obtained from a generator system. A long-lived parent isotope, ⁸²Strontium (⁸²Sr⁺), which has a half-life of 25 days, is immobilized on a so-called ⁸²Sr/⁸²Rb generator, which is comprised of an adsorbent such as Al₂O₃, ZrO₂, TiO₂. and Sb₂O₅ and the like. This tightly binds the ⁸²Sr.

Over time, the immobilized ⁸²Sr⁺ decays to ⁸²Rb⁺, which does not bind tightly to the generator. When the generator is eluted with a physiologically acceptable solution such as a saline solution, the ⁸²Rb⁺ is swept off the column and infused into a patient. Such generators are well known in the art. For example, see Gennaro et al., EP 172106 A1 and Neirinckx et al, Evaluation of inorganic materials as adsorbents for the strontium-82/rubidium-82 generator. Squibb Inst. Med. Res., New Brunswick, N.J., USA. International Journal of Applied Radiation and Isotopes (1983), 34(4), 721-5.

Due to the short half-life of the isotope (75 seconds), an automated infusion system such as that described in U.S. Pat. No. 4,585,009 is typically used. Such an infusion system is able to accurately quantify the amount of ⁸²Rb⁺ infused. Such generators/infusion systems are commercially available under, for example, the tradename CardioGen®, and are described in, for example, U.S. Pat. No. 4,585,009.

For myocardial imaging studies with ⁸²Rb under resting conditions, the patient is positioned under the detector, and a slow or fast bolus of ⁸²Rb⁺ (typically about 40-60 mCi) is infused. Scanning is initiated within 1-2 minutes and continues until most of the ⁸²Rb⁺ has decayed (typically 4-6 minutes). For studies with ²⁰¹Tl⁺, which has a much longer half-life, the isotope is administered via intravenous (IV) injection and a planar or SPECT camera is used to obtain images.

For both agents, in some instances, a second “stress” image is obtained. A second dose of radioisotope is injected along with a vasodilator such as adenosine or dipyrimadole. This increases blood flow significantly. The differences in the images obtained at rest and under such stress conditions can be used to detect the presence of and assess the severity of coronary artery stenosis. Such procedures are known in the art and are described in, for example, Induction of pharmacological stress with alkynyladenosine A2A adenosine receptor agonists. Linden, et al, WO 00/78774

Both ⁸²Rb⁺ and ²⁰¹Tl⁺ are primarily used for myocardial imaging, with some applications in tumor imaging as well. For example, accumulation of ²⁰¹Tl in cancer cells has been reported. ²⁰¹Tl scans have been used to diagnose various types of cancer, to establish its relationship with the proliferation of cancer cells and potentially to predict the response to chemotherapy. However, in these applications, ²⁰¹Tl⁺ is administered by itself and the degree of uptake is generally modest. Tl⁺ is injected, and after a suitable time delay, the tumor is detected by an increased amount of radioactivity in said tumor, relative to the surrounding tissues.

It would be useful to be able to use these nuclear imaging agents for other purposes, as they are already approved for clinical use. In particular, it would be very useful to find applications wherein these isotopes could be used to usefully and reliably image cancer or other diseases, and to obtain information about the presence of receptors on such cells.

Many types of cancer cells, including breast, pancreatic and lung cancers, medullary thyroid cancer cells, neuroendocrine tumors, gastrointestinal adenocarcinomas, lung cancers, cancers of the prostate, gut, kidney, and ovary and brain tumors such as glioma are known to over-express specific receptors, such as gastrin-releasing peptide-, neurotensin-, substance P-, glucagon-like peptide 1-, neuropeptide Y-, somatostatin, cholecystekinin or corticotropin-releasing factor-receptors.

Imaging of such tumors that over express a particular receptor has been accomplished by radiolabeling a receptor binding agent with a label such as ¹¹¹In, ^{99m}Tc, ¹⁷⁷Lu, ¹³¹I and the like. The presence of the cancer is detected when such a radiolabeled receptor binding agent binds to said receptors at concentrations high enough to be visualized relative to background tissues.

For example, it is known that [(¹¹¹)In-DTPA-D-Phe1]-octreotide (DTPA, diethylenetriaminepentaacetic acid), (¹⁷⁷)Lu-DOTA(0), Tyr(3)]octreotate and other somatostatin derivatives bind to somatostatin receptors, [(¹¹¹)In-DTPA-Arg¹]-substance P binds to substance P receptors, (¹¹¹)In-DTPA-D-Glu(1)-minigastrin binds to cholescystokinin (CCK-B)/gastrin receptors, and ¹⁷⁷Lu-derivatives of bombesin bind to GRP and NMB receptors. There is a wide body of literature based on the selective targeting of cancerous tissue with radiolabeled receptor binding agents.

However, for such studies, a different radiolabeled receptor binding compound must be used for each type of receptor. The development of such an agent is expensive and time consuming.

As discussed briefly above, mono-cations such as ⁸²Rb⁺ and ²⁰¹Tl⁺ are recognized as analogs for the potassium ion (K⁺) in vivo. The concentration of potassium ions in cells is typically higher than that outside the cells in the extracellular space. It is known that this concentration gradient is normally maintained by K⁺ delivery systems which include the (Na⁺, K⁺)-ATPase pump and the Na⁺/K⁺/Cl⁻ co-transport systems. K⁺ concentration in cells is also affected by K⁺ efflux systems including K⁺ channels.

Influx of potassium and its analogs is mediated (in part) by the activity of the sodium/potassium ATPase [Na+/K+ ATPase]. This enzyme is known to catalyze the hydrolysis of ATP coupled with the exchange of sodium and potassium ions across the plasma membrane. This action creates an electrochemical gradient of sodium and potassium ions, providing the energy for active transport of various substances.

ATP+H₂O+Na⁺(inside)+K⁺(outside)-→ADP+phosphate+Na⁺(outside)+K⁺(inside)

It has been observed in vitro, in cell culture experiments with living cells and with neurons, that the treatment these cells and neurons with a variety of receptor binding compounds has a dramatic effect on the influx of Rb⁺ that has been placed in the cell media. These studies are frequently performed using an isotope of Rubidium known as ⁸⁶Rb⁺, which is not used clinically. Treatment with these agents causes the ⁸⁶Rb⁺, a mono-cationic tracer, to influx from the media into the cells to levels that are significantly higher than are found in cells that are not treated with said agents.

For example, it has been found that the receptor-binding peptide known as bombesin caused “marked increase” in ⁸⁶Rb⁺ uptake in quiescent Swiss 3T3 cells in vitro. When treated with 6.2 nM bombesin, the initial rate of total ⁸⁶Rb⁺ uptake into the cells was increased by 70±3% (n=49). This uptake was linear for at least 20 min. These cells contain the GRP receptor, to which bombesin binds. Structurally related bombesin analogs such as litorin, GRP(1-27) and neuromedin B had a similar effect.

The marked uptake of Rb⁺ occurred within seconds after the addition of the peptide and appeared to be mediated by an increase in Na⁺ entry into the cells. The authors found that the effect of bombesin was concentration dependent with half-maximal stimulation occurring at 0.3-0.4 nM bombesin.

This stimulation of ⁸⁶Rb⁺ uptake was mediated by the Na⁺/K⁺ pump, since it was virtually abolished by treatment of the cells with a specific inhibitor of the sodium/potassium ATPase pump known as ouabain. The fact that treatment with ouabain could block such uptake showed that the observed influx of Rb⁺ was an energy dependent process that is coupled to the (Na⁺K⁺ ATPAse pump).

It has also been found that stimulation of alpha(1)-adrenoceptors (AR) in isolated perfused rat hearts and isolated cardiomyocytes by the receptor-binding compound phenylephrine (30 μM) increased ⁸⁶Rb⁺ influx into myocardial cells. This increase appeared to be mediated by the Na⁺-K⁺-2Cl⁻ co-transporter.

In studies with human proinsulin C-peptide, a compound which shows specific binding to cell membrane binding sites, incubation of human renal tubular cells with 5 nM human proinsulin C-peptide at 37° for 10 min stimulated ⁸⁶Rb⁺ uptake by 40% (p<0.01). The carboxy-terminal pentapeptide was found to elicit 57% of the activity of the intact molecule.

It has also been shown that treatment of rat L6 skeletal muscle cells in culture with insulin, which binds to insulin receptors, stimulates Na⁺-K⁺-2Cl⁻ cotransporter (NKCC)-mediated ⁸⁶Rb⁺ influx significantly (approximatly 1.6-fold), following acute cell exposure to insulin.

In yet another experiment, myocardial uptake of ⁸⁶Rb in atrial slices in culture was reported to be increased in the presence of 3 nM concentrations of the receptor-binding compound 3,5,3-triiodothyronine (T3).

Platelet-derived growth factor (PDGF) has been reported to cause an acute increase in the influx of ⁸⁶Rb in rat vascular smooth muscle cells in culture.

Bradykinin receptor stimulation by treatment with bradykinin has been shown to stimulate Rb⁺ influx in NIH-3T3 fibroblasts. The furosemide-sensitive Na⁺/K⁺/Cl— cotransporter and the ouabain-sensitive Na⁺/K⁺-ATPase were both involved in Rb⁺ influx under resting conditions. Bradykinin was reported to stimulate Rb⁺ influx (+82.6%) through both systems.

The effect of in vitro treatment of guinea pig pancreatic acinar cells with cholecystekinin octapeptide (CCK-8 or Sincalide), carbachol, forskolin, 8-bromo- and dibutyryl cAMP, theophylline, and isobutylmethylxanthine was investigated. Carbachol (CCh) and cholecystokinin octapeptide (CCK-8) each stimulated ouabain-sensitive ⁸⁶Rb⁺ uptake by approximately 60%. Secretin increased Na⁺-K⁺ pump activity (as measured by ouabain-sensitive ⁸⁶Rb⁺ uptake) by approximately 40% as did forskolin, 8-bromo- and dibutyryl cAMP, theophylline, and isobutylmethylxanthine.

These experiments demonstrate that in vitro treatment of certain cells and tissues with certain receptor binding compounds can stimulate the selective uptake of mono-cations into said cells or tissues.

The in vitro studies above were based on well known techniques; such experiments are frequently performed to determine the effect of drugs on ⁸⁶Rb influx (or efflux), as this provides indirect evidence of the effect these same drugs will have on potassium (K⁺) influx and efflux. ⁸⁶Rb⁺ is used as a surrogate marker for the influx and efflux of K⁺, as it can be readily detected and quantitated, due to its long half-life (18.7 days). The ⁸⁶Rb⁺ used in these experiments, is a long-lived isotope that does not have clinical applications.

### SUMMARY OF THE INVENTION

The invention is directed to a novel method of imaging, using a radioactive mono-cation in combination with a receptor binding ligand that binds to a receptor in a tissue or tissues, thus stimulating the influx of cations into the tissue of interest. The stimulation of cation influx is exploited to transport the radioactive mono-cation such as ⁸²Rubidium (⁸²Rb) (positron imaging) or ²⁰¹Thallium (²⁰¹Tl) (SPECT or planar imaging), which are analogs for the potassium ion (K⁺) in vivo, into the target tissue. By virtue of the radioactivity that is thus localized, said receptors can be imaged.

The receptor binding ligand that stimulates influx of monocations is administered either before or after the cationic radioisotope, or in a preferred embodiment, they are co-administered. The ligand binds to the receptor, initiating an influx of cations, including the cationic radioisotope, allowing imaging of tissue containing the receptor.

Useful isotopes for this purpose include mono-cationic compounds such as ¹³NH₄⁺, and monocationic radioisotopes such as potassium, cesium, thallium and rubidium, including ³⁸K⁺, ⁴²K⁺, ¹³⁴Cs⁺, ¹³⁷Cs⁺, ⁸¹Rb⁺, ⁸²Rb⁺, ^{82m}Rb⁺, and ²⁰¹Tl⁺.

Useful receptor binding ligands that stimulate an influx of cations include, for example, bombesin, bombesin analogs or other GRP receptor binding ligands. Other ligands include a1A adrenoreceptor binding ligands such as S-methylurapidil; receptor binding ligands such as bradykinin and analogs thereof that bind to bradykinin receptors, Type I Fc receptor binding ligands such as IgE; adenosine receptor binding ligands such as CCPA, R-PIA, and NECA; β-adrenoreceptor binding ligands such as adrenaline, isoprenaline, and salbutanol; somatostatin receptor binding ligands such as somatostatin and its derivatives and metal complexes thereof; and muscarinic acetylcholine receptor (mAChR) binding ligands such as muscarinic agonists, platelet derived growth factor (PDGF), insulin, growth hormones such as triiodothyronine (T3), and cholecystekinin and gastrin derivatives that bind to the cholecystokinin receptor, such as cholecystekinin, CCK-8, Sincalide, minigastrin and their derivatives and analogs.

In a preferred embodiment, the receptor binding ligand is an agonist and is internalized within the target cell upon binding. For example, in a preferred embodiment the receptor binding ligand is a GRP receptor agonist such as bombesin, gastrin releasing peptide, neuromedin B or derivatives thereof, including truncated bombesin (BBN) derivatives containing BBN 7-14; a bradykinin receptor agonist; a serotonin receptor agonist a somatostatin receptor agonist, or a cholecystekinin receptor agonist.

In one embodiment, said method can be used to image the presence of suitable receptor-containing cells or tissues in normal organs such as the heart, liver, GI system, kidneys, brain, adrenals, pancreas, lungs, thyroid, liver, ovaries and the like. For example, the presence of GRP receptors in the pancreas could be detected by infusion of both a monocationic radionuclide such as ⁸²Rb⁺ or ²⁰¹Tl⁺ and a GRP-receptor binding ligand such as bombesin or a derivative or fragment thereof, by virtue of the increased ⁸²Rb or ²⁰¹Tl uptake that ensues when said ligand binds to GRP receptors or receptor subtypes in the pancreas.

In another embodiment, said method is used to visualize the presence or absence of receptor binding cells or tissue found in diseases of various organs and organ systems, such as cardiovascular disease (e.g. myocardial ischemia, myocardial infarction, heart failure and the like), infectious and inflammatory diseases, or the presence of receptor binding cells in tumors in a wide variety of cancers. In a preferred embodiment, said method is used to visualize the presence (or absence) of receptor binding tumors and metastases in cancers such as prostate, lung, breast, kidney, thyroid, colon, ovarian, neuroendocrine and pancreatic cancers, and the like. In an especially preferred embodiment, said method is used to detect the presence of prostate cancer by virtue of the GRP receptors that are over-expressed in said disease.

Several compounds are known to affect the energy dependent mechanisms that are responsible for influx of mono-cations into cell and tissues. These compounds serve to either block influx, block efflux or increase efflux of K⁺ and analogs from the cell. They work via a variety of mechanisms. Such compounds could be administered before, during or after administration of the cationic radioisotope and/or the binding ligand in applications where adjusting influx or efflux is desirable. For example, in situations where the absence of said uptake may prove diagnostic, where the subsequent washout rate of the influxed Rb or TI also provides valuable diagnostic information about the cells viability or biochemical state, and/or where dual phase imaging, e.g. ⁸²Rb administered in the absence and presence of the receptor binding agent might give useful differential information.

### DETAILED DESCRIPTION OF THE INVENTION

The invention is directed to a novel method of imaging, using a radioactive monocation such as ⁸²Rubidium (positron imaging) or ²⁰¹Thallium (SPECT or planar imaging), in combination with a receptor binding ligand that stimulates the influx of cations.

The influx of cations caused by the receptor binding ligand is used to transport the radioactive mono-cation into the tissue of interest, which is then imaged using the appropriate type of imaging.

Other useful compounds for this purpose include ¹³NH₄⁺, and radioisotopes of potassium, thallium and rubidium, including ³⁸K⁺, ⁸¹Rb⁺⁸²Rb⁺, ^{82m}Rb⁺ and ²⁰¹Tl⁺. ⁸²Rb⁺ and ²⁰¹Tl are particularly preferred. For positron emitting radioisotopes such a s⁸¹Rb⁺⁸²Rb⁺, ^{82m}Rb⁺ and ¹³NH₄⁺, images may be obtained using Positron Emission Tomography, or PET, as these compounds emit positrons. For radioisotopes of thallium SPECT or planar imaging may be used.

Useful receptor binding ligands which stimulate an influx of cations include, for example, bombesin, bombesin analogs or other GRP receptor binding ligands. Other useful receptor binding ligands include:
a1A adrenoreceptor binding ligands such as S-methylurapidil;
alpha-1-selective agonists such as phenylephrine;
alpha-2-selective agonists such as clonidine;
Bradykinin receptor binding ligands such as Bradykinin;
Type I Fce receptor binding ligands such as IgE;
adenosine receptor binding ligands such as CCPA, R-PIA, NECA;
β-adrenoreceptor binding ligands such as adrenaline, isoprenaline, and salbutanol;
somatostatin receptor binding ligands such as somatostatin and derivatives thereof;
muscarinic acetylcholine receptor (mAChR) binding ligands such as muscarinic agonists;
adrenergic receptor agonists such as phenylephrine, isoproterenol, or epinephrine;
the receptor binding compounds insulin, epidermal growth factor, and prostaglandins (E1 and F2alpha);
Angiotensin II;
5-hydroxytryptamine (5-HT), also known as serotonin and analogs thereof;
Triiodothyronine;
Platelet-derived growth factor;
Insulin,
Cholecystokinin and analogs and derivatives such as CCK-8 or Sincalide.

The list above serves only to exemplify some of the binding ligands that may be useful and is not intended to be limiting. The receptor binding ligand may be a peptide, a polypeptide, a monomer, a dimer, a multimer, a peptidomimetic, a non-peptide, an antibody fragment, an antibody (humanized or non-humanized), a protein, a hormone, a growth factor, a cytokine or a drug. In a preferred embodiment, the receptor binding ligand is peptide, and in an especially preferred embodiment, the peptide is an agonist.

In a preferred embodiment the receptor binding ligand is an agonist and is internalized within the target cell upon binding. For example, in a preferred embodiment the receptor binding ligand is a GRP receptor agonist such as bombesin, gastrin releasing peptide, neuromedin B or derivatives thereof, including truncated bombesin (BBN) derivatives containing BBN 7-14. When administered before, during or after administration of the radioactive cation, administration of a GRP receptor agonist permits imaging of both normal GRP receptor expressing tissue, and in particular GRP receptor expressing tumor tissue such as prostate cancer, breast cancer, gastrinoma, glioblastoma, some small cell lung cancers and uterine tumors.

In another preferred embodiment, the receptor binding ligand is a bradykinin receptor agonist. For example, bradykinin (Arg-Pro-Pro-Gly-Phe-Ser-Pro-Phe-Arg) [BK] serves as a selective agonist for the bradykinin receptor subtype 2, and des-Arg⁹-BK is a specific stimulant of bradykinin receptor subtype 1 [B1R]. Bradykinin and its analogs are an important growth factor for small-cell lung cancer (SCLC) and prostate cancer (PC) and it has been reported that BK receptors are expressed on almost all lung cancer cell lines and on many prostate cancer cells. In addition, whereas the bradykinin receptor subtype 2 [B2R] is constitutively expressed and is thought to mediate most of the physiological actions of kinins, the B1R is generally not expressed under non-pathological conditions but undergoes marked up-regulation after cell injury and stress. When administered before, during or after administration of the radioactive cation, a bradykinin receptor agonist permits imaging of bradykinin receptor expressing tissue, including lung and prostate cancer cells.

In another embodiment, the receptor binding ligand is a serotonin receptor agonist. It has been shown that receptors for serotonin (5-HT) are highly upregulated in some tumors. For example, the 5-HT receptor subtypes 5-HT 1A and 5-HT 1B have been demonstrated on samples of human prostate cancer, in prostate cancer cells that have metastasized to lymph node and bone, and in human prostate cancer cell lines PC-3, DU145, LNCaP. The 5-HT 2A serotoninergic receptor is expressed in the MCF-7 human breast cancer cell line. Serotonin increases uptake of ⁸⁶Rb⁺ in cultured guinea pig tracheal smooth muscle cells. Thus ⁸⁶Rb uptake in serotonin-expressing tumor cells can be increased if said cancer cells were treated with serotonin and analogs. Consequently, administration of a serotonin receptor agonist before, during or after a radioactive cation permits imaging of serotonin receptor containing tissue, particularly a variety of serotonin receptor expressing cancers.

In another preferred embodiment, the receptor binding ligand is a CCK receptor agonist or a gastrin agonist derivative (these two peptides are known to have significant homology). Cholecystekinin receptors are reported to be over expressed in numerous neuroendocrine tumors, including carcinoid tumors, gastrinomas, glucagonomas, insulinomas and paragangiomas, over 90% of all medullary thyroid carcinomas, as well as in stromal tumors and in pancreatic cancer. They are also expressed in normal tissue in the hepatobiliary and GI systems. Thus ⁸⁶Rb uptake in CCK receptor-expressing tumor cells can be increased if said cancer cells are treated with cholecystekinin (CCK), CCK-8, Sincalide or analogs. Consequently, administration of a CCK receptor agonist before, during or after a radioactive cation permits imaging of CCK receptor containing tissue, particularly in neuroendocrine tumors, medullary thyroid carcinoma, and pancreatic cancer, and may prove useful for the imaging of pancreas, gall bladder and GI systems.

Agonist receptor binding agents are preferred because the internalization process that occurs when agonists are bound to their cognate receptor requires energy. Internalization of the occupied receptors can trigger, for example, Na⁺/K⁺ ATPase pump activity or Na⁺/K⁺/Cl— cotransporter activity, depending on the receptor type and cell type involved. When internalization takes place, potassium and its analogs (such as radioactive cations including Rb⁺ or Tl⁺) are also taken into the cell. Thus, radioactive cation uptake, including Rb⁺ or Tl⁺ uptake, has the potential to be an indirect measure of receptor internalization and the degree to which it is coupled to the sodium/potassium pump or transporter systems or other energy requiring processes such as kinase activity.

As such, the method of the invention is useful to ascertain whether receptors are present on tissue(s) of interest and to image said receptor-bearing tissue. In one embodiment, the method of the invention could be used to identify the presence of receptors on normal tissue. In another embodiment, the claimed method could be used to identify receptors in cancerous tissue such as tumors or metastases. Additionally, the claimed methods may be used to image cancer or other diseases which involve receptor-expressing tissue.

In a particularly preferred embodiment, the receptor binding ligand is a GRP receptor agonist such as bombesin or an analog or derivative thereof, including, for example, BBN 7-14.

he receptor binding ligand is administered either before or after the cationic radioisotope, or in a preferred embodiment, they are co-administered. The ligand binds to the receptor, initiating an influx of cations, including the cationic radioisotope, allowing imaging of tissue containing the receptor.

For example, in one preferred embodiment, bombesin or a bombesin analog or derivative is administered either before or with ⁸²Rb infusion. Preferably they are co-administered. The bombesin binds to GRP expressing tissue, including for example, GRP expressing tumors, initiating an influx of cations, including the ⁸²Rb which had been administered. The ⁸²Rb internalized in the tissue of interest is imaged using positron emission tomography. The resulting images can be used to diagnose the presence of such GRP receptor positive tumors. For GRP receptors, such tumors include prostate, breast, gastric and lung cancers as well as glioblastoma. This method could also be used for the imaging of normal tissue that expresses GRP-r, such as pancreas.

An advantage of the present invention is that unlike imaging studies with a radiolabeled receptor binding ligand, such as bombesin, it is not the binding of the radiolabeled ligand that is being measured, but the influx of Rb⁺. For radiolabeled receptor binding ligands, it is possible to administer them in such quantities that substantially all receptors become occupied. At this point, no further binding of radioactive receptor binding ligand is possible. For radiolabeled compounds with a low specific activity, this can be a real problem. With the current invention the likelihood of saturation caused by an excess of binding ligand at the receptor would be reduced, as it is the resultant Tl⁺ or Rb⁺ uptake that is being measured.

Additionally, the method of the invention potentially exposes the patient to less radiation exposure then, for example, use of a beta-emitting radiolabeled receptor binding ligand (e.g. a receptor binding ligand labeled with ¹⁷⁷Lu). Thus, the method of the invention could be especially useful in the diagnosis of early stage cancers, such as early stage prostate cancer where the use of a beta-emitting receptor binding compound such as a beta-emitting bombesin analog, might be contraindicated.

The two key components of this invention, the receptor binding ligand and the mono-cationic radionuclide may be sold either together or separately. The receptor binding ligand may be provided as a freeze-dried solid that is reconstituted with a physiologically acceptable solution prior to administration, or may be provided in a physiologically acceptable aqueous or non-aqueous solution, in the presence of such buffers, stabilizers, and solubilizers as are necessary to prepare a stable solution of said receptor binding ligand. Prior to administration, the solid or solution may stored frozen or at room temperature, depending on the stability requirements of the compound.

The radioisotope solution will preferably be provided as a physiologically acceptable aqueous solution such as in normal saline. In general, from about 1 to about 60 mCi should be administered. For ²⁰¹Tl, quantities of about 1 to about 5 mCi, preferably 2-3 mCi will be used in a volume of from 0.5 to about 10 mL. For ⁸²Rb, quantities of from about 20 to about 60 mCi, preferably 40 to 60 mCi will be administered in each infusion in a volume of from about 1 to about 30 mL. ⁸²Rb will most preferably be delivered from an ⁸²Sr/⁸²Rb generator coupled to an infusion system, such as is known in the art and discussed above.

The invention also includes “kits” comprising a receptor binding ligand of interest to be used in conjunction with existing radioactive cation products for imaging various targets of interest. For example, for imaging GRP positive tumors, or for imaging of human pancreas, which has demonstrated marked uptake of radiolabeled GRP derivatives, a GRP receptor binding ligand such as bombesin or a derivative thereof could be sold as a kit to be used in conjunction with ⁸²Rb from a separately supplied generator or with existing Tl products. Likewise, for imaging of CCK receptor positive tumors or other CCK positive organs, the commercially available CCK-8 containing product known as Sincalide could be sold for use in conjunction with ⁸²Rb from a separately supplied generator or with existing Tl products.

The receptor binding ligand may be administered before, during or after administration of monocationic radioisotope such as ⁸²Rb or ²⁰¹Tl. Preferably when ⁸²Rb is used, it will be co-administered with the receptor binding ligand. When ²⁰¹Tl is used co-administration is also preferable.

It may be advantageous to administer more than one dose of the monocationic radioisotope such as ⁸²Rb or ²⁰¹Tl. In one embodiment, a radioactive monocation such as ⁸²Rb or ²⁰¹Tl is administered, and an image is obtained using a camera that is capable of detecting the distribution of said monocation. After a suitable amount of time, which will depend on the half-life of the radioisotope and its clearance characteristics, a second dose of the radioactive monocation is administered, either before, at the same time as or following the administration of the receptor binding agent. Alternatively, two monocation administrations and imaging sessions, the first using a radioactive monocation plus a receptor binding agent, and a second administration and imaging session in the absence of the receptor binding agent may be performed. Thus, two sets of images are obtained, one of which shows the distribution of the monocation in the absence of the receptor binding agent, the other of which shows its distribution in the presence of the receptor binding agent. The two images may be compared, or one image may be subtracted from the other to provide a third image that has been corrected for any uptake of monocation that is not receptor mediated.

If there is little or no difference between the two sets of images it may indicate that the receptors under study are not present on the organs that are imaged. If there is a substantial difference between the two images, the difference may indicate an over-expression of such receptors in the organs where a difference is found. Such information may prove useful e.g. to select patients who are candidates for radiotherapeutic treatment with receptor binding radiopharmaceuticals, or to select patients who are candidates for drug treatments with non-radioactive drugs that act on their targets through receptor-mediated pathways, such as anticancer drugs or drugs for the treatment of myocardial diseases.

Depending on the particular application, the receptor binding ligands may be administered orally, sublingually, by intravenous, subcutaneous or intraperitoneal injection. The radioactive monocation may be administered by intravenous, subcutaneous or intraperitoneal injection. For both the receptor binding ligand and the radioisotope, intravenous injection is preferred. Depending on the particular application, the isotope may be injected as a bolus or may be infused slowly. For ⁸²Rb, bolus injection is preferred due to the short half-life of the isotope.

In some instances, the absence of monocation uptake may prove diagnostic. In other instances, the subsequent washout rate of the influxed Rb or Ti may also provides valuable diagnostic information about the cells viability or biochemical state. Alternatively, dual phase imaging is also envisioned, e.g. ⁸²Rb administered in the absence and presence of the receptor binding agent might give useful differential information.

Several compounds are known to affect the energy dependent mechanisms that are responsible for influx of mono-cations into cell and tissues. These compounds serve to block influx, block efflux or increase efflux of K⁺ and analogs from the cell. They work via a variety of mechanisms. Such compounds could be administered before, during or after administration of the cationic radioisotope and/or the binding ligand in applications where adjusting or controlling influx or efflux is desired.

For example, ouabain, a cardiac glycoside, is known to inhibit the Na⁺/K⁺ ATPase pump, resulting in decrease in the uptake of Rb⁺, K⁺ and analogs, and in some cases, an increase in K⁺ efflux from the cells. The structure of oubain is:

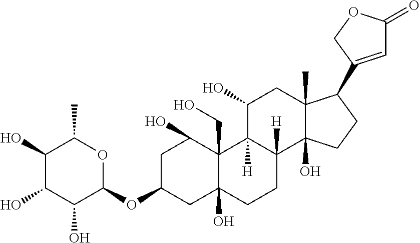

In some instances, it might be useful to confirm that the enhanced uptake of a mono-cation such as ⁸²Rb⁺ into tissues following treatment with a receptor binding agonist is, in fact due to the binding of said receptor binding agent to said receptor on the target tissues. This could be confirmed by repeating the binding studies in the presence of ouabain or an ouabain-like compound, if receptor binding and Rb uptake are coupled to one another via the Na⁺/K⁺ ATPase pump.

Other compounds increase the efflux of Rb⁺, K⁺ and analogs. For example, treatment of cells with Ca⁺² ionophores such as A23187 (which facilitates entry of Ca⁺² into the cell) may increase the rate of efflux of ⁸⁶Rb⁺ and ⁸²Rb⁺ from the cell. The structure of A23187 is:

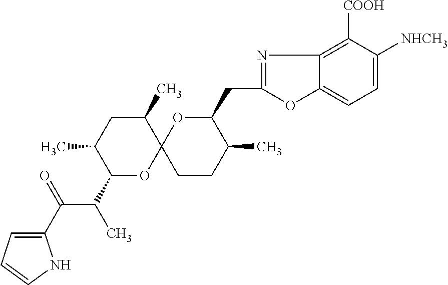

Likewise, increasing Ca²⁺ may have a similar effect. Agents which stimulate or activate Ca⁺² channels, such as Bay K8644, thus stimulating the influx of Ca⁺² and raising intracellular Ca⁺² levels, may increase the rate of efflux of mono-cations such as ²⁰¹Tl and ⁸²Rb. The structure of Bay K8644 is:

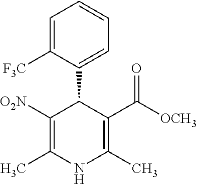

Still other compounds decrease the rate of efflux from cells. For example, the binding of apamin, CNCKAPETALCARRCQQH-NH₂ (a bee venom polypeptide) to apamin receptors on cells is known to block Ca²⁺-activated K⁺ channels, and to decrease the rate of Rb⁺ efflux from such cells.

Likewise, the treatment of certain cells with tetraethyl ammonium (TEA) ion, (Et)₄N⁺ or nitrendipine, 2,6-Dimethyl-3-acetyl-5-carbomethoxy-4-(4′-methoxyphenyl)-1,4-dihydropyridine, may also inhibit Ca²⁺-activated K⁺ channels and thus inhibit Rb⁺ efflux. Such agents, which inhibit Ca⁺² channels, may be used to reduce the rate of efflux of mono-cations such as ⁸⁶Rb⁺, ⁸²Rb⁺ and 20Tl⁺.

Finally, some agents can stimulate efflux (and/or block uptake). Such agents may be used for example, for evaluating the efficacy of potential new drugs that are designed to block receptors in the heart, GI or other organs (e.g. evaluation of new beta blockers etc.) Preferred embodiments of the invention include:

A method of imaging receptor-expressing tissue comprising:
i. Administering a radioactive mono-cation;
b. Administering a binding ligand which binds the desired receptor and stimulates influx of cations; and
c. Imaging the receptor-expressing tissue using a camera capable of detecting the radioactive mono-cation.
   The method of imaging of embodiment 1 wherein steps a and b occur simultaneously.
   The method of imaging of embodiment 1 wherein step b occurs before step a.
   The method of imaging of embodiment 1 wherein step a occurs before step b.

1. A method of imaging receptor-expressing tissue comprising:
a. administering a radioactive mono-cation;
b. administering a binding ligand which binds the desired receptor and stimulates influx of cations; and
c. imaging the receptor-expressing tissue using a camera capable of detecting the radioactive mono-cation.

2. The method of imaging of claim 1 wherein steps a and b occur simultaneously.

3. The method of imaging of claim 1 wherein step b occurs before step a.

4. The method of imaging of claim 1 wherein step a occurs before step b.

5. The method of claim 1 wherein the mono-cation is selected from the group consisting of ¹³NH₄⁺, and monocationic radioisotopes such as radioactive potassium, cesium, thallium and rubidium.

6. The method of claim 5 wherein the mono-cation is selected from the group consisting of ⁴²K⁺, ⁸¹Rb⁺, ^{82m}Rb⁺, ⁸²Rb⁺, ⁸⁶Rb⁺, and ²⁰¹Tl⁺.

7. The method of claim 6 wherein the mono-cation is selected from the group consisting of ⁸²Rb⁺, ⁸⁶Rb⁺ and ²⁰¹Tl⁺.

8. The method of claim 1, wherein the binding ligand is selected from the group consisting of bombesin, bombesin analogs, other GRP receptor binding ligands, a1A adrenoreceptor binding ligands, receptor binding ligands that bind to bradykinin receptors, Type I Fc receptor binding ligands, adenosine receptor binding ligands, β-adrenoreceptorbinding ligands, somatostatin receptor binding ligands, cholescystokinin receptor binding ligands, and muscarinic acetylcholine receptor (mAChR) binding ligands.

9. The method of claim 8, wherein the binding ligand is selected from bombesin, bombesin(7-14), neuromedin B, litorin and derivatives thereof, S-methylurapidil; bradykinin; IgE; CCPA; R-PIA; NECA; adrenaline; isoprenaline; salbutanol; somatostatin, octreotide, DTPA-D-Phe¹]-octreotide, DOTA-TATE, and derivatives or analogs thereof, cholescystekinin, cholescystekinin(1-8) CCK8, Sincalide and derivatives thereof, muscarinic agonists, platelet derived growth factor (PDGF), insulin, and growth hormones.

10. The method of any one claims 1 or 8, wherein the binding ligand is an agonist and is internalized within the target cell upon binding.

11. The method of claim 8, wherein the binding ligand is a GRP receptor agonist.

12. The method of claim 11, wherein the binding ligand is a BBN 7-14.

13. The method of claim 8 wherein the receptor binding ligand is cholecystekinin, CCK8 or Sincalide.

14. The method of claim 8, wherein the receptor binding ligand is octreotide, DTPA-D-Phe¹]-octreotide or DOTA-TATE.

15. The method of claim 1, wherein the receptor-expressing tissue is found in normal tissue.

16. The method of claim 1, wherein the receptor-expressing tissue is found in diseased tissue.

17. The method of claim 1, further comprising administering a compound which affects the influx or efflux of K⁺ or analogs from the cell.

18. The method of claim 17, wherein the compound blocks the influx, blocks the efflux or increases the efflux of K⁺ or analogs from the cell.

19. The method of claim 17, wherein administration of the compound which affects the influx or efflux of K⁺ or analogs from the cell occurs before or during administration of the cationic radioisotope and/or the binding ligand.

20. The method of claim 17, wherein the compound which affects the influx or efflux of K⁺ or analogs from the cell is selected from the group consisting of ouabain, A23187, Ca²⁺, Bay K8644, apamin, tetraethyl ammonium (TEA) ion, and nitrendipine.

21. A method of imaging GRP receptor-expressing tissue comprising:
a. administering ⁸²Rb⁺;
b. administering an agonist binding ligand selected from the group consisting of bombesin, bombesin analogs or other GRP receptor binding ligands which binds the GRP receptor and stimulates influx of cations; and
c. imaging the receptor-expressing tissue using a camera capable of detecting the ⁸²Rb⁺.

22. The method of claim 21, wherein the ⁸²Rb⁺ and the binding ligand are co-administered.

23. The method of claim 21, wherein the GRP receptor-expressing tissue is normal tissue.

24. The method of claim 21, wherein the GRP receptor-expressing tissue is diseased tissue.

25. A method for diagnosing the presence of GRP receptor positive tumors in a patient with such tumors comprising:
a. administering ⁸²Rb⁺;
b. administering a binding ligand which binds the GRP receptor on GRP receptor positive tumors and stimulates influx of cations, selected from the group consisting of bombesin, bombesin analogs or other GRP receptor binding ligands which are agonists; and
c. imaging the receptor-expressing tissue using a camera capable of detecting the ⁸²Rb⁺.

26. The method of claim 25, wherein the ⁸²Rb⁺ and the binding ligand are co-administered.

27. The method of any one of claims 21 or 25, further comprising administering a compound which blocks the influx, blocks the efflux or increases the efflux of K+ or analogs from the cell.

28. The method of claim 1, further comprising the steps of i) administering a radioactive cation, and ii) imaging the tissue using a camera capable of detecting the radioactive mono-cation, prior to performing steps a-c, and comparing the image obtained in step ii to the image obtained in step c.

29. The method of claim 1, further comprising the steps of:
d. administering a radioactive mono-cation after steps a-c have been completed;
e. imaging the receptor-expressing tissue using a camera capable of detecting the radioactive mono-cation; and
f. comparing the images obtained in steps c and e.

30. The method of any one of claims 21 or 25, further comprising the steps of i) administering ⁸²Rb⁺ and ii) imaging the tissue using a camera capable of detecting the ⁸²Rb⁺, prior to performing steps a-c, and comparing the image obtained in step ii to the image of the GRP receptor-expressing tissue obtained in step c.

31. The method of any one of claims 21 or 25, further comprising the steps of:
d. administering ⁸²Rb⁺ after steps a-c have been completed;
e. imaging the GRP receptor-expressing tissue using a camera capable of detecting the radioactive mono-cation; and
f. comparing the images obtained in steps c and e.
